(19) United States
(12) Patent Application Publication
(10) Pub. No.: US 2026/0115131 A1
(43) Pub. Date: Apr. 30, 2026

(54) METHODS OF ADMINISTERING AN ARIPIPRAZOLE INJECTABLE PREPARATION

(71) Applicant: Otsuka Pharmaceutical Co., Ltd., Tokyo (JP)

(72) Inventors: Matthew Harlin, Severna Park, MD (US); Xiaofeng WANG, Ellicott City, MD (US); Yanlin WANG, Jersey City, NJ (US); Arash RAOUFINIA, McLean, VA (US)

(73) Assignee: Otsuka Pharmaceutical Co., Ltd., Tokyo (JP)

(21) Appl. No.: 18/959,760

(22) Filed: Nov. 26, 2024

Related U.S. Application Data
(63) Continuation of application No. 18/184,135, filed on Mar. 15, 2023, now abandoned.
Application No. 18/184,135 is a continuation of application No. 17/594,292, now abandoned.
(60) Provisional application No. 63/003,544, filed on Apr. 1, 2020.

(51) Int. Cl. A61K 9/00; A61K 31/496; A61P 25/18

(52) U.S. Cl. CPC A61K 9/0019; A61K 9/0053; A61K 31/496; A61P 25/18

(57) ABSTRACT

The present disclosure is directed to the methods of treating a patient with schizophrenia or bipolar I disorder by administering to the patient an injectable preparation of aripiprazole, wherein the patient is administered the injectable preparation about once every two months.

### TECHNICAL FIELD

The present application claims priority to U.S. Provisional Application No. 63/003,544, filed Apr. 1, 2020; the content of this application is incorporated by reference herein in its entirety.

### BACKGROUND ART

Aripiprazole, a partial agonist at the dopamine (D2) and serotonin 5-HT1A receptors, and an antagonist at serotonin 5-HT2A receptors, is an atypical antipsychotic that has demonstrated efficacy in clinical trials for the treatment of schizophrenia and bipolar I disorder in adults. Aripiprazole is 7-[4-[4-(2,3-dichlorophenyl)-1-piperazinyl]butoxy]-3,4-dihydrocarbostyril. The empirical formula is C₂₃H₂₇Cl₂N₃O₂ and its molecular weight is 448.38. The chemical structure is:

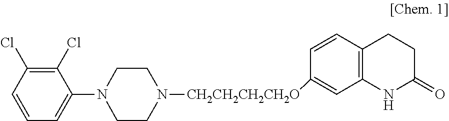

A pharmaceutical composition comprising aripiprazole is known as an antipsychotic useful for the treatment of schizophrenia. Abilify Maintena®, the intramuscular (IM) depot formulation of aripiprazole, is a prolonged-release suspension for injection. It is approved in many countries for the maintenance treatment of schizophrenia and bipolar I disorder in adult patients stabilized with oral aripiprazole.

Currently approved by the US Federal Drug Administration, Abilify Maintena® for intramuscular injection encompasses an extended-release suspension for monthly administration at dosages of 160 mg, 200 mg, 300 mg, and 400 mg of aripiprazole or a salt thereof. Those compositions and methods are described in U.S. Pat. Nos. 7,807,680, 8,030,313, 8,338,427, 8,338,428, 8,399,469, 8,722,679, 8,759,351, 8,993,761, 9,089,567, and 10,525,057; all of which are incorporated herein by reference in their entirety.

Other formulations of long-acting compositions of aripiprazole encompass the use of an injectable preparation comprising aripiprazole or salt thereof, the crystalline form of aripiprazole or a salt thereof, a specific suspending agent (suspending agent (A)), and a dispersion medium. Those injectable preparations are known as ready to use (RTU) formulations of aripiprazole; they are described at least in U.S. Pat. No. 10,517,951, which is incorporated herein by reference in its entirety.

### SUMMARY OF INVENTION

### Technical Problem

The RTU injectable preparations of aripiprazole allow for the administration in patient populations considered to be at potential risk for adherence-related relapse or suboptimal treatment outcomes and achieving therapeutic plasma concentrations without the reliance on daily oral tablet dosing. That is, the present disclosure is directed to the use of the RTU injectable preparation of aripiprazole to minimize one or more of relapse, suboptimal treatment outcome, and/or achieving therapeutic plasma concentration. For example, the present disclosure is directed to methods of treating a patient with schizophrenia or bipolar I disorder by administering intramuscularly to the patient an injectable preparation comprising aripiprazole or a salt thereof wherein the patient is administered the injectable preparation about once every two months. Administration of the injectable preparation about once every two months may improve medication compliance.

### Solution to Problem

In some aspects, the present disclosure is directed to a method of treating a patient with schizophrenia or bipolar I disorder comprising: administering intramuscularly to the patient an injectable preparation comprising aripiprazole or a salt thereof, wherein the patient is administered the injectable preparation about once every two months. With the methods disclosed herein, the injectable preparation comprises an amount of aripiprazole ranging from about 650 mg to about 1200 mg. Additionally, with the methods disclosed herein, administering intramuscularly to the patient is at a site chosen from a deltoid muscle, a gluteal muscle, and combinations thereof, for example at a gluteal muscle.

In further aspects, the methods provided herein the injectable preparation further comprises: water, and a suspending agent comprising polyvinylpyrrolidone, polyethylene glycol, and carboxymethylcellulose or a salt thereof, wherein aripiprazole or a salt thereof has a mean primary particle diameter ranging from about 0.5 μm to about 30 μm and the concentration of aripiprazole or a salt thereof ranges from about 200 mg/mL to about 600 mg/mL. Further for example, with the methods disclosed herein one or more injectable preparations are administered.

In aspects of the present disclosure, a method of treating a patient with schizophrenia or bipolar I disorder comprising: administering to the patient at a gluteal muscle an injectable preparation comprising aripiprazole or a salt thereof, wherein the patient is administered the injectable preparation about once every 56 days (8 weeks, or 2 months), and the amount of aripiprazole in the injectable preparation ranges from about 650 mg to about 1200 mg.

In some further aspects, the present disclosure is directed to a method of treating a patient with schizophrenia or bipolar I disorder comprising: administering to the patient at a gluteal muscle an injectable preparation comprising aripiprazole or a salt thereof, wherein the patient is administered the injectable preparation about once every 56 days, and wherein the injectable preparation further comprises: water, and a suspending agent comprising polyvinylpyrrolidone, polyethylene glycol, and carboxymethylcellulose or a salt thereof, wherein the concentration of aripiprazole in the injectable preparation is 300 mg/mL.

In some further aspects, the present disclosure is directed to aripiprazole or a salt thereof for use in the treatment of schizophrenia or bipolar I disorder, wherein aripiprazole or a salt thereof is to be administered to a patient in need thereof with the methods disclosed herein. Additionally, the present disclosure also provides use of aripiprazole or a salt thereof in the manufacture of a medicament for treating schizophrenia or bipolar I disorder, wherein aripiprazole or a salt thereof is to be administered to a patient in need thereof with the methods disclosed herein.

### BRIEF DESCRIPTION OF DRAWINGS

FIG. 1 is directed to the mean (SD) pharmacokinetic concentrations following the first dose of the investigational medicinal product being aripiprazole 2 month (2M) RTU (referenced as “Ari 2M group”) at 960 mg (N=41) (1 injection for 56 days) with oral administration of aripiprazole for consecutive 7 days from the first day of the first administration of Ari 2M RTU, and the reference component was aripiprazole intramuscular depot (referenced as “Ari IM depot” or “IM depot”) at 400 mg (N=42) (1 injection for 28 days), Abilify Maintena®, with oral administration of aripiprazole for consecutive 14 days from the first day of the first administration of Ari IM depot.

FIG. 2 is directed to the mean (SD) pharmacokinetic concentrations following the 4^{th} dose of Ari 2M at 960 mg (N=102) of aripiprazole (56-day intervals) compared to IM depot at 400 mg (N=93) of aripiprazole (28-day intervals) following the 7th and 8th doses.

FIG. 3 diagrams mean (SD) aripiprazole plasma concentration time profiles following administration of a single dose of 780 mg (N=18) or 1200 mg (N=13) aripiprazole 2M RTU long acting injectable ready to use to the gluteal muscle of subjects with schizophrenia.

### DESCRIPTION OF EMBODIMENTS

As used herein, “a” or “an” entity refers to one or more of that entity, e.g., “a compound” refers to one or more compounds or at least one compound unless stated otherwise. As such, the terms “a” (or “an”), “one or more”, and “at least one” are used interchangeably herein.

As used herein, the term “about” means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 5%.

As used herein, the term “treat,” “treating,” or “treatment,” when used in connection with a disorder or condition, includes any effect, e.g., lessening, reducing, modulating, ameliorating, or eliminating, that results in the improvement of the disorder or condition. Improvements in or lessening the severity of any symptom of the disorder or condition can be readily assessed according to standard methods and techniques known in the art. In some embodiments, the presently disclosed methods can be used to treat schizophrenia and bipolar I disorder, as maintenance monotherapy. In further embodiments, the presently disclosed methods can be used to treat schizophrenia, acute treatment of manic and mixed episodes associated with Bipolar I disorder, major depressive disorder (MDD), irritability with Autistic Disorder, and Tourette's disorder.

As used herein, reference to “aripiprazole” is to aripiprazole or a salt thereof, the crystalline form of aripiprazole or a salt thereof. Aripiprazole or a salt thereof may be in a monohydrate form (aripiprazole hydrate A) or in various anhydrous forms, which are known to exist in the form of anhydrous crystal B, anhydrous crystal C, anhydrous crystal D, anhydrous crystal E, anhydrous crystal F, and anhydrous crystal G. All of these crystalline forms may be used as aripiprazole or a salt thereof in the injectable preparation of the present disclosure and further for example, aripiprazole is a monohydrate form. As used herein, the term “aripiprazole” or salt thereof refers to a compound having the structure:

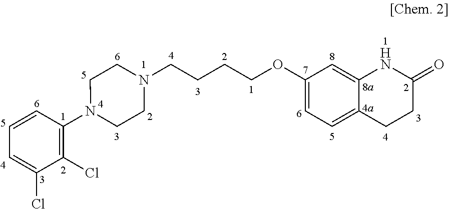 [SMILES: O=C1CCc2ccc(OCCCCN3CCN(c4cccc(Cl)c4Cl)CC3)cc2N1]

The present disclosure is directed to the methods of treating a patient with schizophrenia or bipolar I disorder by administering intramuscularly to the patient an injectable preparation of aripiprazole, wherein the patient is administered the injectable preparation about once every two months.

### EMBODIMENTS

Without limitation, some embodiments of the disclosure include:

1. A method of treating a patient with schizophrenia or bipolar I disorder comprising: administering intramuscularly to the patient an injectable preparation comprising aripiprazole or a salt thereof, wherein the patient is administered the injectable preparation about once every two months.

2. The method according to embodiment 1, wherein the administration of the injectable preparation results in a total amount of aripiprazole ranging from about 650 mg to about 1200 mg to the patient every two months.

3. The method according to embodiment 2, wherein the administration of the injectable preparation results in the total amount of aripiprazole of about 960 mg to the patient every two months.

4. The method according to any one of embodiments 1 to 3, wherein the about once every two months comprises about once every 42 days to 70 days.

5. The method according to embodiment 4, wherein the once every two months comprises once every about 56 days (e.g., once every 54 days to 58 days).

6. The method according to any one of embodiments 1 to 5, wherein the patient has schizophrenia.

7. The method according to any one of embodiments 1 to 5, wherein the patient has bipolar I disorder.

8. The method according to any one of embodiments 1 to 7, wherein administering intramuscularly to the patient is at a site chosen from a deltoid muscle, a gluteal muscle, and combinations thereof.

9. The method according to embodiment 8, wherein the site is the gluteal muscle.

10. The method according to any one of embodiments 1 to 9, wherein the injectable preparation further comprises: water, and a suspending agent comprising polyvinylpyrrolidone, polyethylene glycol, and carboxymethylcellulose or a salt thereof, wherein aripiprazole or a salt thereof has a mean primary particle diameter ranging from about 0.5 μm to about 30 μm and the concentration of aripiprazole or a salt thereof ranges from about 200 mg/mL to about 600 mg/mL.

11. The method according to any one of embodiments 1 to 9, wherein the injectable preparation further comprises: water for injection, and a suspending agent comprising polyvinylpyrrolidone, polyethylene glycol, and carboxymethylcellulose or a salt thereof, wherein the aripiprazole or a salt thereof is aripiprazole monohydrate, the aripiprazole monohydrate has a mean primary particle diameter ranging from about 2 μm to about 5 μm, and the concentration of aripiprazole in the injectable preparation is about 300 mg/mL which is calculated based on the anhydrous form of aripiprazole.

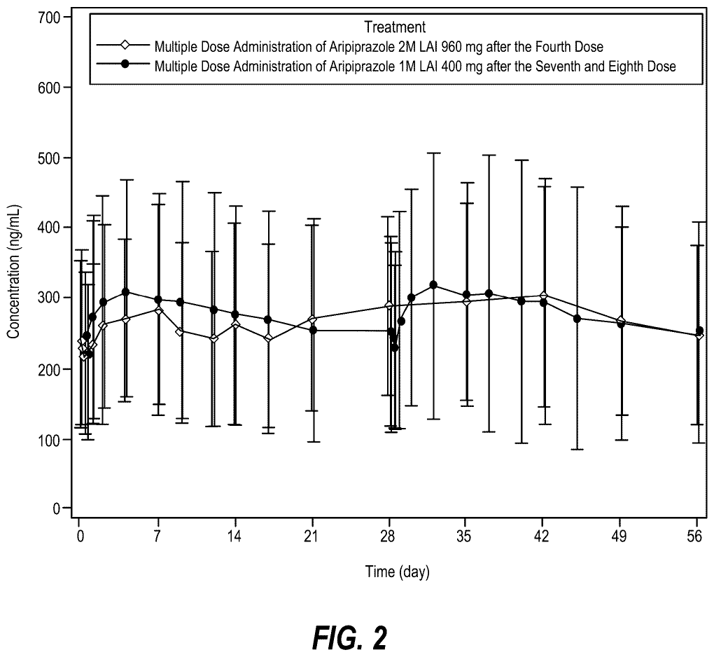

12. The method according to any one of embodiments 1 to 11, wherein an area under the concentration-time curve (AUC) of aripiprazole from time zero to 56 days postdose following a plurality of administration of the injectable preparation is substantially equal to a twofold AUC of aripiprazole from time zero to 28 days postdose following a plurality of administration of an aripiprazole intramuscular (IM) depot formulation such as Abilify Maintena® about once every one month (4 weeks or 28 days), e.g., as shown in FIG. 2.

13. The method according to any one of embodiments 1 to 11, further comprising administering orally to the patient a solid dosage form comprising aripiprazole or a salt thereof, wherein the injectable preparation and the solid dosage form are administered to the patient on a first day, and the solid dosage form is administered for consecutive 5 to 15 days from the first day.

14. A method of treating a patient with schizophrenia or bipolar I disorder comprising: administering intramuscularly to the patient an injectable preparation comprising aripiprazole or a salt thereof, wherein the patient is administered the injectable preparation about once every two months and wherein administering to the patient intramuscularly is at a site chosen from a deltoid muscle, a gluteal muscle, and combinations thereof.

15. A method of treating a patient with schizophrenia or bipolar I disorder comprising: administering intramuscularly to the patient an injectable preparation comprising aripiprazole or a salt thereof, wherein the patient is administered the injectable preparation about once every two months, wherein administering intramuscularly to the patient is at a site chosen from a deltoid muscle, a gluteal muscle, and combinations thereof, and wherein the about once every two months comprises about once every 42 days to 70 days.

16. A method of treating a patient with schizophrenia or bipolar I disorder comprising: administering intramuscularly to the patient an injectable preparation comprising aripiprazole or a salt thereof, wherein the patient is administered the injectable preparation about once every two months, wherein administering intramuscularly to the patient is at a site chosen from a deltoid muscle, a gluteal muscle, and combinations thereof, wherein the about once every two months comprises about once every 42 days to 70 days, and wherein the administration of the injectable preparation results in a total amount of aripiprazole ranging from about 650 mg to about 1200 mg to the patient every two months.

17. A method of treating a patient with schizophrenia or bipolar I disorder comprising: administering intramuscularly to the patient an injectable preparation comprising aripiprazole or a salt thereof, wherein the patient is administered the injectable preparation about once every two months, wherein administering intramuscularly to the patient is at a site chosen from a deltoid muscle, a gluteal muscle, and combinations thereof, wherein the about once every two months comprises about once every 42 days to 70 days, wherein the administration of the injectable preparation results in a total amount of aripiprazole ranging from about 650 mg to about 1200 mg to the patient every two months, and wherein the injectable preparation further comprises: water, and a suspending agent comprising polyvinylpyrrolidone, polyethylene glycol, and carboxymethylcellulose or a salt thereof, wherein aripiprazole or a salt thereof has a mean primary particle diameter ranging from about 0.5 μm to about 30 μm and the concentration of aripiprazole or a salt thereof ranges from about 200 mg/mL to about 600 mg/mL.

18. A method of treating a patient with schizophrenia or bipolar I disorder comprising: administering to the patient at a gluteal muscle an injectable preparation comprising aripiprazole or a salt thereof, wherein the patient is administered the injectable preparation about once every 56 days (8 weeks or 2 months), and the amount of aripiprazole in the injectable preparation ranges from about 650 mg to about 1200 mg.

19. The method according to embodiment 18, wherein the injectable preparation further comprises: water, and a suspending agent comprising polyvinylpyrrolidone, polyethylene glycol, and carboxymethylcellulose or a salt thereof, wherein aripiprazole or a salt thereof has a mean primary particle diameter ranging from about 0.5 μm to about 30 μm, and the concentration of aripiprazole in the injectable preparation is about 300 mg/mL.

20. The method according to embodiment 18 or 19, wherein the injectable preparation further comprises: water for injection, and a suspending agent comprising polyvinylpyrrolidone, polyethylene glycol, and carboxymethylcellulose or a salt thereof, wherein the aripiprazole or a salt thereof is aripiprazole monohydrate, the aripiprazole monohydrate has a mean primary particle diameter ranging from about 2 μm to about 5 μm, and the concentration of aripiprazole in the injectable preparation is about 300 mg/mL which is calculated based on the anhydrous form of aripiprazole.

21. The method according to any one of embodiments 18 to 20, wherein an area under the concentration-time curve (AUC) of aripiprazole from time zero to 56 days postdose following a plurality of administration of the injectable preparation is substantially equal to a twofold AUC of aripiprazole from time zero to 28 days postdose following a plurality of administration of an aripiprazole intramuscular (IM) depot formulation such as Abilify Maintena® about once every one month (4 weeks or 28 days), e.g., as shown in FIG. 2.

22. The method of according to any one of embodiments 18 to 20, further comprising administering aripiprazole in a solid dosage form orally, and the solid dosage form is administered in an amount in a range of 10 mg to 20 mg per day for consecutive 5 to 15 days with the first administration of the injectable preparation.

23. The method according to any one of embodiments 18 to 20, further comprising administering 10 mg to 20 mg of aripiprazole in a solid dosage form orally per day for consecutive 7 days with the first administration of the injectable preparation.

24. A method of treating a patient with schizophrenia or bipolar I disorder comprising: administering to the patient at a gluteal muscle an injectable preparation comprising aripiprazole or a salt thereof, wherein the patient is administered the injectable preparation about once every 56 days (8 weeks or two months), the injectable preparation is administered in an amount of aripiprazole ranging from about 650 mg to about 1200 mg to the patient, and wherein the injectable preparation further comprises: water, and a suspending agent comprising polyvinylpyrrolidone, polyethylene glycol, and carboxymethylcellulose or a salt thereof, wherein aripiprazole or a salt thereof has a mean primary particle diameter ranging from about 0.5 μm to about 30 μm.

25. The method of according to embodiment 24, further comprising administering aripiprazole in a solid dosage form orally, and the solid dosage form is administered in an amount in a range of 10 mg to 20 mg per day for consecutive 5 to 15 days with the first administration of the injectable preparation.

26. The method according to embodiment 24, further comprising administering 10 to 20 mg of aripiprazole in a solid dosage form orally per day for consecutive 7 days with the first administration of the injectable preparation.

27. A method of administering an injectable preparation comprising aripiprazole or a salt thereof to a patient with schizophrenia or bipolar I disorder comprising:
administering orally to the patient 10 to 20 mg/day of aripiprazole or a salt thereof for consecutive 5 to 15 days from a first day;
administering intramuscularly to the patient the injectable preparation once every about 56 days (every about 8 weeks or two months),
wherein the injectable preparation comprising about 650 mg to 1200 mg of aripiprazole is administered on the first day, and a series of the administrations allows the mean plasma level of aripiprazole to maintain 90 ng/mL or more, for example, at least for about 56 days.

28. The method according to embodiment 27, wherein the method does not comprise a further oral administration of aripiprazole or a salt thereof.

29. A method of administering a prefilled sol-gel formed injectable preparation comprising aripiprazole or a salt thereof to a patient in need thereof, comprising:
administering 50 to 300 mg of aripiprazole, as a total amount, simultaneously or consecutively from a first day to the 15th day or less; and
administering the prefilled sol-gel formed injectable preparation comprising 650 to 1200 mg of aripiprazole at a gluteal muscle on the first day; followed by
administering the prefilled sol-gel formed injectable preparation comprising about 650 mg to 1200 mg of aripiprazole at a gluteal muscle once every about 56 days (every about 8 weeks or two months),
wherein a series of the administrations allows the mean plasma level of aripiprazole to maintain 90 ng/mL or more.

30. The method according to embodiment 29, wherein the prefilled sol-gel formed injectable preparation is an injectable preparation comprising 300 mg/mL of aripiprazole, having a thixotropic property.

31. The method according to embodiment 29 or 30, wherein the injectable preparation further comprises a suspending agent selected from the group consisting of polyvinylpyrrolidone, polyethylene glycol, carboxymethylcellulose or a salt thereof, and any combination thereof.

32. A method of administering a long acting injectable preparation comprising aripiprazole or a salt thereof to a patient in need thereof, comprising:
administering to the patient the injectable preparation comprising 650 to 1200 mg of aripiprazole at a gluteal muscle once every about 56 days (every about 8 weeks or two months),
wherein the patient has a stable plasma level of aripiprazole by being subject to administration of aripiprazole but is in need of additional administration of aripiprazole, the injectable preparation is in a dosage form of a prefilled sol-gel formed injection, and a series of the administrations allows the mean plasma level of aripiprazole to maintain 90 ng/mL or more.

33. The method according to embodiment 32, wherein the stable plasma level of aripiprazole is 90 ng/mL or more.

34. A method of treating a patient with schizophrenia or bipolar I disorder comprising: administering intramuscularly to the patient an injectable preparation comprising aripiprazole or a salt thereof about once every two months (8 weeks or 56 days), wherein an area under the concentration-time curve (AUC) of aripiprazole from time zero to 56 days postdose following a plurality of administration of the injectable preparation is substantially equal to a twofold AUC of aripiprazole from time zero to 28 days postdose following a plurality of administration of an aripiprazole intramuscular (IM) depot formulation such as Abilify Maintena® about once every one month (4 weeks or 28 days), e.g., as shown in FIG. 2.

35. The method according to embodiment 34, wherein the aripiprazole or a salt thereof is aripiprazole monohydrate.

36. The method according to embodiment 34 or 35, wherein the plurality of administration of the injectable preparation is the 4th or more of the administration, and the plurality of administration of the aripiprazole intramuscular (IM) depot formulation is the 7th or more of the administration.

37. A method of treating a patient with schizophrenia or bipolar I disorder comprising: administering intramuscularly to the patient an injectable preparation comprising aripiprazole or a salt thereof about once every two months (8 weeks or 56 days), wherein the plasma concentration of aripiprazole in 56 days postdose following a plurality of administration of the injectable preparation is substantially equal to the plasma concentration of aripiprazole in 28 days following a plurality of administration of an aripiprazole intramuscular (IM) depot formulation such as Abilify Maintena® about once every one month (4 weeks or 28 days), e.g., as shown in FIG. 2.

38. The method according to embodiment 37, wherein the aripiprazole or a salt thereof is aripiprazole monohydrate.

39. The method according to embodiment 37 or 38, wherein the plurality of administration of the injectable preparation is the 4th or more of the administration, and the plurality of administration of the aripiprazole intramuscular (IM) depot formulation is the 7th or more of the administration.

### Injectable Preparation

An injectable preparation of the present disclosure (e.g., two month ready to use (RTU) injectable preparation), comprises a composition comprising aripiprazole, a specific suspending agent (suspending agent (A)), and a dispersion medium. For example, the injectable preparation of the present disclosure comprises at least water as a dispersion medium. Water, or an aqueous solvent comprising water and an organic solvent can be used as a dispersion medium comprising at least water. In some aspects, the dispersion medium is water, and further for example, sterile water for injection.

The specific suspending agent (suspending agent A) contained in the injectable preparation of the present disclosure comprises at least one suspending agent chosen from (i) and (ii) (i) polyvinylpyrrolidone, and (ii) polyethylene glycol and carboxymethyl cellulose or a salt thereof.

In some aspects, the injectable preparation of the present disclosure comprises aripiprazole or a salt thereof, water, and at least one suspending agent chosen from groups (i) and (ii): (i) polyvinylpyrrolidone, and (ii) polyethylene glycol and carboxymethylcellulose or a salt thereof, wherein aripiprazole or a salt thereof has a mean primary particle diameter ranging from about 0.5 μm to about 30 μm and the concentration of aripiprazole or a salt thereof ranges from about 200 mg/mL to about 600 mg/mL. The presently disclosed, two month ready to use (RTU), injectable preparation is described in U.S. Pat. No. 10,517,951, which is incorporated herein by reference in its entirety.

For example, when the injectable preparation of the present disclosure comprises aripiprazole or a salt thereof (which hereafter may be referred to as “the aripiprazole injectable preparation of the present disclosure”) the concentration of aripiprazole or a salt thereof ranges from about 200 mg/mL to about 600 mg/mL. (α) The RTU injectable preparations of aripiprazole become a gel upon standing, which precipitation and caking of the particles of aripiprazole can be inhibited, thereby providing excellent storage stability. Furthermore, because (β) the injectable preparation, even in the form of a gel, can easily gain fluidity when subjected to a mild impact, the preparation can be easily injected at the time of use (at the time of injection). Further, because the gelled injectable preparation (gel composition) gains fluidity (forms a sol state) by simply pressing the plunger of a syringe and ejecting the preparation through a syringe needle, the preparation can be smoothly ejected through the needle as is. Therefore, the preparation can have a thixotropic property, and be well dispersed intramuscularly or subcutaneously with relatively less local disturbance and pain at the time of injection.

That is, when the concentration thereof is 100 mg/mL or below, the production of an injectable preparation that can form a gel may not occur even if the suspending agent A is used (or an aging treatment is further performed). Therefore, the injectable preparation of the present disclosure comprises aripiprazole or a salt thereof, a combination of the use of a specific suspending agent (suspending agent A) and a specific concentration of aripiprazole or a salt thereof (from about 200 mg/mL to about 600 mg/mL, such as from about 250 mg/mL to about 450 mg/mL, and from about 300 mg/mL to about 400 mg/mL) is used. When the injectable preparation of the present disclosure comprises a salt of aripiprazole, the concentration described above is calculated as aripiprazole.

In some aspects, the RTU injectable preparation comprises aripiprazole in a concentration of about 200 mg/mL to about 600 mg/mL, such as about 200 mg/mL to about 500 mg/mL, for example, about 250 mg/mL to about 450 mg/mL, further for example, about 300 mg/mL to about 400 mg/mL, and further for example, about 300 mg/mL.

In some aspects, in the aripiprazole injectable preparation of the present disclosure, when (i) polyvinylpyrrolidone is contained as suspending agent A, the concentration of polyvinylpyrrolidone ranges from about 0.1 mg/mL to about 100 mg/mL, such as about 1 mg/mL to about 50 mg/mL, and further for example about 2 mg/mL to about 20 mg/mL.

In some aspects, when the aripiprazole injectable preparation of the present disclosure comprises (i) polyvinylpyrrolidone as suspending agent A, and further comprises one or more other suspending agents, at least one member is chosen from polyethylene glycol and carboxymethylcellulose or a salt thereof be contained as the one or more other suspending agents. For example, these injectable preparations of the present disclosure comprise (i) polyvinylpyrrolidone as suspending agent A, and when they further comprise one or more other suspending agents, they comprise suspending agents of any combination of (i-1) to (i-3) shown below.
(i-1) polyvinylpyrrolidone and polyethylene glycol
(i-2) polyvinylpyrrolidone and carboxymethylcellulose or a salt thereof, and
(i-3) polyvinylpyrrolidone, polyethylene glycol, and carboxymethylcellulose or a salt thereof.

Regardless of which combination of (i-1) to (i-3) these injectable preparations of the present disclosure comprise, the concentration of polyvinylpyrrolidone is, as described above, about 0.1 mg/mL to about 100 mg/mL, such as about 1 mg/mL to about 50 mg/mL, and further for example, about 2 mg/mL to about 20 mg/mL. In (i-1) or (i-3), the concentration of polyethylene glycol is about 0.05 mg/mL to about 100 mg/mL and for example, about 0.1 mg/mL to about 50 mg/mL. In (i-2) or (i-3), the concentration of carboxymethylcellulose or a salt thereof is about 0.5 mg/mL to about 50 mg/mL, such as about 1 mg/mL to about 30 mg/mL, and further for example, about 2 mg/mL to about 20 mg/mL.

By containing carboxymethylcellulose or a salt thereof, an increase in viscosity during production can be suppressed. This allows aripiprazole or a salt thereof, to be pulverized into a desirable particle size in an efficient manner. In some embodiments, the molecular weight of carboxymethylcellulose or a salt thereof ranges from 49,000 to 300,000. Furthermore, by containing polyethylene glycol, syneresis can be prevented even when the resulting injectable preparation is stored for a long period of time. In some embodiments, the molecular weight of polyethylene glycol ranges from 400 to 4,000. In some embodiments, (i-3) is present in the injectable preparation.

In some aspects, when the aripiprazole injectable preparation of the present disclosure comprises (ii) polyethylene glycol and carboxymethylcellulose or a salt thereof as suspending agent A, the concentration of polyethylene glycol is about 0.05 mg/mL to about 2 mg/mL and further for example, about 0.1 mg/mL to about 1 mg/mL. The concentration of carboxymethylcellulose or a salt thereof is about 0.5 mg/mL to about 50 mg/mL, such as about 1 mg/mL to about 30 mg/mL, and such as about 2 mg/mL to about 20 mg/mL.

In some aspects, when the aripiprazole injectable preparation of the present disclosure comprises (ii) polyethylene glycol and carboxymethylcellulose or a salt thereof as suspending agent A, and further comprises one or more other suspending agents, polyvinylpyrrolidone can be contained as the one or more other suspending agents. For example, the injectable preparation of the present disclosure comprises, as suspending agent A, (ii) polyethylene glycol and carboxymethylcellulose or a salt thereof, and when it further comprises one or more other suspending agents, the suspending agents of (i-3) are contained. In this case, the concentrations of polyethylene glycol, carboxymethylcellulose or a salt thereof, and polyvinylpyrrolidone are the same as described in (i-3) above.

In some aspects, in the aripiprazole injectable preparation of the present disclosure, when the suspending agents of (i-3) are used, the composition comprises about 0.5 mg/mL to about 20 mg/mL of polyvinylpyrrolidone, about 0.1 mg/mL to about 100 mg/mL of polyethylene glycol, about 0.5 mg/mL to about 50 mg/mL of carboxymethylcellulose or a salt thereof, and about 250 mg/mL to about 450 mg/mL (such as about 300 mg/mL to about 400 mg/mL) of aripiprazole or a salt thereof. In this case, the polyethylene glycol may be polyethylene glycol 400 or polyethylene glycol 4000. In some aspects, the polyvinylpyrrolidone has a K value of about 12 to about 20. In further aspects, the aripiprazole or a salt thereof has a mean primary particle diameter of about 1 μm to 10 μm.

Because an unduly large mean primary particle diameter of the aripiprazole or a salt thereof, may cause precipitation, the mean primary particle diameter ranges from about 0.5 μm to about 30 μm and for example, about 1 μm to about 20 μm. When the injectable preparation of the present disclosure is in a dosage form that is administered once every two months, the aripiprazole or a salt thereof has a mean primary particle diameter of about 1 μm to about 50 μm, such as about 1 μm to about 10 μm, and further for example, about 2 μm to about 5 μm. The mean secondary particle diameter is up to but not exceeding three times and further for example, up to but not exceeding twice the mean primary particle diameter.

In some aspects, the injectable preparation of the present disclosure is suitably formulated into a dosage form that can be administered once every two months. For example, the concentration of aripiprazole or a salt thereof in the injectable preparation of the present disclosure that is administered once every two months, is, calculated as aripiprazole, about 200 mg/mL to about 600 mg/mL, such as about 250 mg/mL to about 500 mg/mL, further for example, about 300 mg/mL to 400 mg/mL, and further for example, about 300 mg/mL. When the injectable preparation is administered once every two months, the dosage volume is about 2 mL to about 4 mL, such as about 2.2 mL to about 3.5 mL, further for example, about 2.3 mL to about 3.4 mL, and further for example, about 3.2 mL.

In some aspects, the aripiprazole injectable preparation of the present disclosure achieves the effects (α) and (β) described above. They may be in the form of a gel or they may have fluidity (i.e., they may be in the form of a sol). As described above, the achievement of the effects of the effects (α) and (β) can objectively confirmed by the use of a rotary rheometer. The injectable preparation may be formulated into a dosage form of a prefilled sol-gel formed injection (also herein referred to as a “prefilled sol-gel formed injectable preparation”). This injectable preparation exhibits a thixotropic property. And the preparation may be in the form of a gel when allowed to stand, and may change to a sol when subjected to an impact.

In some aspects, a method for producing the aripiprazole injectable preparation comprises preparing a liquid mixture of the starting materials and pulverizing aripiprazole or a salt thereof, contained in the liquid mixture to a desired mean primary particle diameter, optionally followed by aging.

In some aspects, a method for producing the gel aripiprazole injectable preparation according to the present invention comprises allowing a liquid mixture to stand at 5 to 70° C. for 5 minutes or more, the liquid mixture comprising aripiprazole or a salt thereof with a mean primary particle diameter of about 0.5 μm to about 30 μm in a concentration of about 200 mg/mL to about 600 mg/mL, water, and at least one suspending agent chosen from (i) and (ii): (i) polyvinylpyrrolidone and (ii) polyethylene glycol and carboxymethyl cellulose or a salt thereof.

For example, in some further aspects, a production method comprising the following steps can be used: pulverizing aripiprazole or a salt thereof to a mean primary particle diameter of about 0.5 μm to about 30 μm in a liquid mixture comprising the aripiprazole or a salt thereof in a concentration of about 200 mg/mL to about 600 mg/mL, water, and at least one suspending agent selected from the group consisting of (i) and (ii): (i) polyvinylpyrrolidone and (ii) polyethylene glycol and carboxymethyl cellulose or a salt thereof; and allowing the pulverized liquid mixture to stand at 5° C. to 70° C. for 5 minutes or more.

In some aspects, the present disclosure comprises the administration of the aripiprazole injectable preparation resulting in a total amount of aripiprazole ranging from about 650 mg to about 1200 mg, for example from about 690 mg to about 960 mg, further for example from about 750 mg to about 960 mg, administered to the patient, wherein the injectable preparation may be optionally administered in one or more divided injections. In some further aspects, the administration of the aripiprazole injectable preparation results in a total amount of aripiprazole of about 960 mg to the patient, wherein one or more injectable preparations are administered. For example, to obtain a total amount of 960 mg of aripiprazole administered to the patient, one (1) injection of the aripiprazole injectable preparation comprising 300 mg/mL of aripiprazole is administered. The 960 mg dosage may be optionally administered in separate injections at short intervals. The number of injections and the concentration of the aripiprazole varies in view of the total amount of aripiprazole to be administered and the concentration of aripiprazole contained in each of the injectable preparations, as described above.

In some aspects, the administration of the aripiprazole injectable preparation to the subject is injected intramuscularly. In some embodiments, administering intramuscularly is at a site chosen from a deltoid, a gluteal muscle, and combinations thereof. In some embodiments, the site is the gluteal muscle. For example, depending on the number of injections for administration, the injection site may encompass various locations of the deltoid and/or gluteal muscles.

In some aspects, the methods of administering the injectable preparation of aripiprazole disclosed herein occur at a frequency of about once every two months. For example, the injectable preparation is administered about once every 42 to 70 days or at any integer in between and including the end points, e.g., every 49 to 63 days, e.g., every 50 to 60 days, i.e., 50, 51, 52, 53, 54, 55, 56, 57, 58, 59 or 60 days. In further aspects, the injectable preparation is administered about once every 56 days, e.g., once every 54 days to 58 days. In further aspects, the injectable preparation is administered about once every 8 weeks, e.g., once every 6 weeks to 10 weeks.

In some aspects, the methods disclosed herein comprise administering orally to the patient 10 to 20 mg/day of aripiprazole or a salt thereof for about consecutive 5 to 15 days, for example about consecutive 7 to 14 days, further for example about consecutive 7 days, from a first day of administration, and administering intramuscularly to the patient an injectable preparation comprising aripiprazole or a salt thereof once every about 56 days (every 8 weeks or two months), wherein the injectable preparation comprising about 650 mg to 1200 mg of aripiprazole is administered on the first day. A series of the administrations allows the mean plasma level of aripiprazole to maintain 90 ng/mL or more. For example, the oral administration may be implemented in a solid dosage form comprising 10 to 20 mg/day of aripiprazole or a salt thereof, and the solid dosage form may be administered for consecutive 5 to 15 days, for example for consecutive 7 days to 14 days, and further for example for consecutive 7 days. Further, for example, the solid dosage form may be administered to the patient in a total amount of 50 mg to 300 mg, 60 mg to 280 mg, 70 mg to 280 mg, or 70 mg to 140 mg of aripiprazole.

In some aspects, the solid dosage form of the present disclosure is an oral (aripiprazole) tablet. In some embodiments, oral tablets of aripiprazole are available in, e.g., 2 mg, 5 mg, 10 mg, 15 mg, and 20 mg strengths, and are available at a single oral dose chosen from 10 mg to 20 mg. In some embodiments, oral tablets of aripiprazole may include inactive ingredients. Inactive ingredients in oral tablets, e.g., include cornstarch, hydroxypropyl cellulose, lactose monohydrate, magnesium stearate, and microcrystalline cellulose. Colorants can include, e.g., ferric oxide (yellow or red) and FD&C Blue No. 2 Aluminum Lake.

In some aspects, the injectable preparation of the present disclosure can be filled as is into a syringe for use as a prefilled syringe. This simplifies the structure of the syringe and reduces size and weight. When the injectable preparation of the present disclosure is filled into a syringe, in a further embodiment, a sol suspension can be administered by simply pressing the plunger rod of the syringe and ejecting the injectable preparation of the present disclosure through a syringe needle. This provides a prefilled syringe that offers clinical convenience and operability, thus is highly useful medically and industrially. An example of producing such a prefilled syringe is such that an injectable preparation is produced in the manner as described above, the preparation is prefilled into a syringe, and then left to stand in the manner as described above to cause the injectable preparation to gel. The present disclosure also includes a kit equipped with the above-described prefilled syringe.

### EXAMPLES

### Clinical Pharmacology Trials

### Example One

This was a phase 1b, open-label, multiple dose, randomized, parallel arm, safety, tolerability, and pharmacokinetic trial of aripiprazole intramuscular depot administered in the gluteal muscle in adult subjects with schizophrenia or bipolar I disorder. Treatment was over 32 weeks with the investigational medicinal product being aripiprazole 2 month (2M) RTU (referenced as “Ari 2M group”) at 960 mg (1 injection for 56 days) and the reference component was aripiprazole intramuscular depot (referenced as “Ari IM depot” or “IM depot”) at 400 mg (1 injection for 28 days), Abilify Maintena®. The primary endpoints were (1) safety and tolerability (e.g., adverse events (AEs), vitals, ECG, labs, physical exams, electrophysiological study (EPS), VAS, investigators assessment of injection, and C-SSR; and (2) pharmacokinetic, e.g., similarity of plasma concentration on last day of dosing internal following the final dose and similarity of aripiprazole exposure (AUC) over the dosing interval following the final dose. The secondary endpoints where PK and efficacy post administration of 2M RTU 960 mg and IM depot 400 mg, respectively.

### Aripiprazole 2 Month (2M) RTU

The 2 month (2M) RTU injectable preparation was supplied as aripiprazole IM depot 300 mg/mL ready-to-use single-dose injection by Otsuka pharmaceuticals Co., Ltd. The injectable preparation comprised aripiprazole monohydrate, carboxymethyl cellulose sodium (5 mg/mL), povidone K17 (4 mg/mL), polyethylene glycol 400 (1 mg/mL), sodium phosphate monobasic monohydrate (0.74 mg/mL), sodium chloride (6.1 mg/mL), sodium hydroxide (pH adjuster), and water for injection. The concentration of aripiprazole in the injectable preparation was 300 mg/mL. This concentration was calculated based on the anhydrous form of aripiprazole.

The 2 month (2M) RTU long-acting injectable (LAI) preparation was administered to an adult subject (N=41) at 56-day (±2 days) intervals with 300 mg/mL of aripiprazole in the injectable preparation (3.2 mL/injection) over the course of 32 weeks (total of 4 injections), and administration occurred at a gluteal site of the subject. 10 to 20 mg of aripiprazole was orally administered in a tablet per day for consecutive 7 days from the first day of the first administration of the 2M RTU injectable preparation.

### Aripiprazole Intramuscular Depot (Reference)

The aripiprazole intramuscular (IM) depot formulation was supplied as a single-dose lyophilized 400 mg strength vial. The labeled strengths were calculated based on the anhydrous form (aripiprazole). In some embodiments, inactive ingredients (per administered dose) for 400-mg strength product include carboxymethyl cellulose sodium (16.64 mg), mannitol (83.2 mg), sodium phosphate monobasic monohydrate (1.48 mg) and sodium hydroxide (pH adjuster). The extended-release injectable suspension in 400-mg strength vial can be used to make dosage adjustments; that is, in patients who are CYP2D6 poor metabolizers and in patients taking concomitant CYP3A4 inhibitors or CYP2D6 inhibitors. Dosage adjustments for 200 mg and 160 mg can be obtained by using the 400-mg strength vial for intramuscular deltoid or gluteal injection for patients taking CYP2D6 inhibitors, CYP3A4 inhibitors, or CYP3A4 for greater than 14 days.

The aripiprazole intramuscular (IM) depot formulation (400-mg strength) was administered to an adult subject (N=42) at 28-day (±2 days) intervals over the course of 32 weeks (total of 8 injections). 10 to 20 mg of aripiprazole was orally administered in a tablet per day for consecutive 14 days from the first day of the first administration of the IM depot formulation.

Disposition: As noted in Tables 2 and 3, the subjects' baseline characteristics were balanced across treatment groups, i.e., diagnosis, gender, race/ethnicity, age, and body mass index (BMI) were similar. Discontinuation rates (Table 1) were lower in the Aripiprazole 2M group (23%) compared to the IM depot group (31%). The reason for discontinuation in both treatment groups were withdrawal by subject, adverse event, and lost to follow-up.

TABLE 1
Subject Disposition

| | Aripiprazole 2M RTU 960 mg | | | Aripiprazole IM Depot 400 mg | | |
|---|---|---|---|---|---|---|
| | Robust | Sparse | All | Robust | Sparse | All |
| Randomized | 42 (100.0) | 90 (100.0) | 132 (100.0) | 42 (100.0) | 92 (100.0) | 134 (100.0) |
| Completed | 36 (85.7) | 66 (73.3) | 102 (77.3) | 34 (81.0) | 58 (63.0) | 92 (68.7) |
| Discontinued | 6 (14.3) | 24 (26.7) | 30 (22.7) | 8 (19.0) | 34 (37.0) | 42 (31.3) |
| Adverse Event | 1 (2.4) | 4 (4.4) | 5 (3.8) | 2 (4.8) | 8 (8.7) | 10 (7.5) |
| Lost to | 1 (2.4) | 2 (2.2) | 3 (2.3) | 1 (2.4) | 6 (6.5) | 7 (5.2) |
| Follow-up | | | | | | |
| Protocol | 1 (2.4) | 1 (1.1) | 2 (1.5) | 1 (2.4) | 1 (1.1) | 2 (1.5) |
| Deviation | | | | | | |
| Withdrawal | 3 (7.1) | 13 (14.4) | 16 (12.1) | 4 (9.5) | 14 (15.2) | 18 (13.4) |
| by Subject | | | | | | |
| Physician | 0 (0.0) | 1 (1.1) | 1 (0.8) | 0 (0.0) | 1 (1.1) | 1 (0.7) |
| Decision | | | | | | |
| Other: | 0 (0.0) | 2 (2.2) | 2 (1.5) | 0 (0.0) | 3 (3.3) | 3 (2.2) |
| COVID-19 | | | | | | |
| (non-AE) | | | | | | |
| Other: Not | 0 (0.0) | 1 (1.1) | 1 (0.8) | 0 (0.0) | 1 (1.1) | 1 (0.7) |
| due to | | | | | | |
| COVID-19 | | | | | | |

TABLE 2
Subject Demographic Characteristics

| | Aripiprazole 2M RTU 960 mg | | | Aripiprazole IM Depot 400 mg | | |
|---|---|---|---|---|---|---|
| | Schizophrenia | Bipolar | All | Schizophrenia | Bipolar | All |
| Randomized | 92 | 40 | 132 | 93 | 41 | 134 |
| Age (mean) | 48.1 | 47.2 | 47.8 | 47.7 | 44.8 | 46.8 |
| Sex (% Female) | 27 (29.3) | 15 (37.5) | 42 (31.8) | 26 (28.0) | 22 (53.7) | 48 (35.8) |
| BMI (kg/m²) | 28.2 | 28.1 | 28.2 | 29.1 | 27.2 | 28.6 |
| Race (%) | | | | | | |
| White | 11 (12.0) | 18 (45.0) | 29 (22.0) | 12 (12.9) | 21 (51.2) | 33 (24.6) |
| Black or | 80 (87.0) | 19 (47.5) | 99 (75.0) | 77 (82.8) | 18 (43.9) | 95 (70.9) |
| African- | | | | | | |
| American | | | | | | |
| Ethnicity (%) | | | | | | |
| Hispanic or | 4 (4.3) | 8 (20.0) | 12 (9.1) | 4 (4.3) | 7 (17.1) | 11 (8.2) |
| Latino | | | | | | |
| Not Hispanic | 87 (94.6) | 32 (80.0) | 119 (90.2) | 88 (94.6) | 34 (82.9) | 122 (91.0) |
| or Latino | | | | | | |

TABLE 3
Subject Baseline Disease Characteristics (Mean (SD))

| | Aripiprazole 2M RTU 960 mg | | | Aripiprazole IM Depot 400 mg | | |
|---|---|---|---|---|---|---|
| | Schizophrenia | Bipolar | All | Schizophrenia | Bipolar | All |
| Randomized | 92 | 40 | 132 | 93 | 41 | 134 |
| SWN-S | 94.3 (16.4) | 92.1 (17.2) | 93.7 (16.6) | 95.6 (15.6) | 88.6 (18.6) | 93.6 (16.8) |
| Total Score | | | | | | |
| PANSS | 62.0 (13.5) | n/a | n/a | 61.8 (13.5) | n/a | n/a |
| TOTAL | | | | | | |
| SCORE | | | | | | |
| CGI- | 3.3 (0.9) | n/a | n/a | 3.1 (0.9) | n/a | n/a |
| Severity | | | | | | |
| Score | | | | | | |
| YMARS | n/a | 6.7 (7.3) | n/a | n/a | 9.4 (8.2) | n/a |
| Total Score | | | | | | |
| MADRS | n/a | 10.9 (9.4) | n/a | n/a | 13.5 (9.7) | n/a |
| Total Score | | | | | | |
| CGI-BP | n/a | | n/a | n/a | | n/a |
| Severity | | | | | | |
| Mania | n/a | 1.8 (1.0) | n/a | n/a | 2.3 (1.2) | n/a |
| Depression | n/a | 2.2 (1.2) | n/a | n/a | 2.5 (1.1) | n/a |
| Overall BP | n/a | 2.4 (1.1) | n/a | n/a | 2.8 (1.2) | n/a |
| Illness | | | | | | |

SWN-S: Subjective Well-being under Neuroleptic Treatment Short Form; PANNS: Positive and Negative Syndrome Scale; CGI-Severity Score: Clinical Global Impression-Severity Score; YMARS: Young Mania Rating Scale; MADRS: Montgomery-Asberg Depression Rating Scale; CGI-BP: Clinical Global Impression-Bipolar Version

Safety: In this clinical trial, the most frequent adverse event was weight increased (22.7% Ari 2M compared to 20.9% Ari IM depot). Injection site pain was the second most frequently reported adverse event (18.2% Ari 2M compared to 9.0% Ari IM depot). The Visual Analog Scale (VAS) perception of pain and the Investigator assessment of injection site found mild and stable throughout the study, which was comparable between treatment groups. Incidence of serious treatment-emergent adverse events (TEAEs) were similar for Ari 2M group (4.5%) and IM depot (6.0%). Fewer subjects in the Ari 2M group (3.0%) discontinued the investigational medicinal product (IMP) due to adverse events compared to those in the IM depot group (7.5%). There were no notable differences observed for both groups in lab, electrocardiogram (ECG) and vital sign except the rates of new onset of QT internal corrected for great rate using Fridericia's formula (QTc) >450 ms for Ari 2M group (0.7%) lower than that for the IM depot group (4.5%).

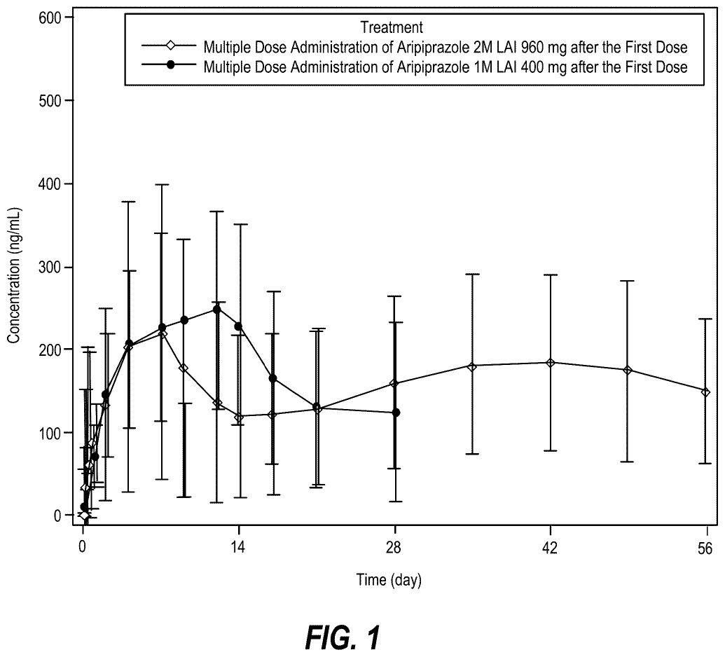

Pharmacokinetic: The pharmacokinetic data is illustrated in FIGS. 1 and 2. In FIG. 1, it charts the mean (SD) pharmacokinetic concentrations following the first administration of Ari 2M at 960 mg of aripiprazole with the oral administration of aripiprazole for consecutive 7 days, compared to IM depot at 400 mg of aripiprazole with the oral administration of aripiprazole for consecutive 14 days. In Table 4 below, summarizes the mean (standard deviation—SD) plasma concentration (ng/mL) of Ari 2M at 960 mg and IM depot at 400 mg at days 7 and 14.

TABLE 4: Comparison of plasma concentration (ng/mL) of Ari 2M at 960 mg and IM depot at 400 mg at days 7 and 14.

TABLE 4

| | | Mean (SD) | Mean (SD) |
|---|---|---|---|
| | N | Day 7 | Day 14 |
| 2M 960 mg | 41 | 221 (178) | 119 (98) |
| IM 400 mg | 42 | 227 (113) | 229 (121) |

In FIG. 2, it charts the mean (SD) pharmacokinetic concentrations following the 4^{th} dose of Ari 2M at 960 mg of aripiprazole compared to IM depot at 400 mg of aripiprazole following the 7^{th} and 8^{th} doses.

Finally, Tables 5 and 6 below provide a summary of the PK parameters and primary PK endpoints, respectively.

TABLE 5
Pharmacokinetic Parameters

| | Aripiprazole | Aripiprazole | Aripiprazole |
|---|---|---|---|
| | 2M LAI 960 mg | IM Depot 400 mg | IM Depot 400 mg |
| PK Parameter | Fourth Dose | Seventh Dose | Eighth Dose |
| Cₘₐₓ (ng/mL) | 342 (157)^{b} | 339 (168)^{d} | 344 (212)^{f} |
| tₘₐₓ (day)^{a} | 28.0 (0.930-49.0)^{b} | 6.97 (1.05-28.0)^{d} | 4.07 (0.00-28.0)^{f} |
| AUC₀₋₅₆ | 14700 (7460)^{b} | ND | ND |
| (ng · day/mL) | | | |
| AUC₀₋₂₈ | 7190 (3470)^{b} | 7760 (4300)^{d} | 7840 (5170)^{f} |
| (ng · day/mL) | | | |
| AUC₂₉₋₅₆ | 7500 (4200)^{b} | ND | ND |
| (ng · day/mL) | | | |
| PTF % | 63.4 (25.1)^{b} | ND | 48.3 (19.0)^{f} |
| C₂₈ (ng/mL) | ND | 255 (137)^{e} | 257 (162)^{g} |
| C₅₆ (ng/mL) | 250 (128)^{c} | ND | ND |

^{a}Median (min-max).
^{b}n = 34.
^{c}n = 96.
^{d}n = 33.
^{e}n = 88.
^{f}n = 32.
^{g}n = 82.

TABLE 6
Pharmacokinetic Parameters

| | PK | | | |
|---|---|---|---|---|
| | Parameter | GMR | 90% CI | P-value |
| Aripiprazole 2M | AUC^{a} | 1.006^{c} | 0.851-1.190 | 0.0129 |
| LAI 960 mg (T) | C₅₆/C₂₈^{b} | 1.011^{d} | 0.893-1.145 | 0.0011 |
| versus | Cₘₐₓ^{b} | 1.071^{c} | 0.903-1.270 | 0.0029 |
| Aripiprazole IM | | | | |
| Depot 400 mg (R) | | | | |

^{a}AUC₀₋₅₆ following the fourth administration of aripiprazole 2M LAI 960 mg or the sum of AUC₀₋₂₈ following the seventh and eighth administration of aripiprazole IM depot 400 mg.
^{b}Following the fourth administration of aripiprazole 2M LAI 960 mg or the eighth administration of aripiprazole IM depot 400 mg.
cn = 34 aripiprazole 2M LAI 960 mg, 32 aripiprazole IM depot 400 mg.
^{d}n = 96 aripiprazole 2M LAI 960 mg, 82 aripiprazole IM depot 400 mg.

1: GMR (AUC₀₋₅₆ after Ari 2M RTU 960 mg 4th injection/sum of AUC₀₋₂₈ after Ari IM depot 400 mg 7th and 8th injections) is derived from ANOVA for log-transformed PK parameters including treatment formulation and disease population as fixed effects. GMR refers to a geometric means ratio.

2: GMR (C₅₆ after Ari 2M RTU 960 mg 4th injection/C₂₈ after Ari IM depot 400 mg 8th injection) is derived from ANOVA for log-transformed PK parameters including treatment formulation, PK sampling schedule and disease population as fixed effects. C56 refers to a plasma concentration of aripiprazole 56 days postdose. C₂₈ refers to a plasma concentration of aripiprazole 28 days postdose.

3: Derived from the one-sided t-test of treatment formulation within the specific ANOVA above to test null hypothesis of ratio of PK parameter for ARI 2M RTU 960 mg to PK parameter for Ari IM depot 400 mg<=0.8.

Efficacy: SWN-S, PANSS, MADRS, and YMRS total scores showed no statistically significant difference between Ari 2M and IM depot groups. Additionally, SWN-S and PANSS showed numerical improvement in change from baseline results for the Ari 2M group for schizophrenia subjects. Overall, the treatment effects of Ari 2M group are comparable to IM depot for both schizophrenia and bipolar I disorder subjects.

### Summary of Efficacy Results and Conclusions:

Subjects with schizophrenia remained stable at low PANSS and CGI-S scores for the duration of the trial in both treatment groups, with no clinically meaningful differences between the treatment groups for the duration of the trial.
No clinically meaningful difference was demonstrated between the 2 treatment groups for the MADRS total score or YMRS total score for subjects with bipolar I disorder.
The mean change from baseline for the CGI-BP severity of illness score for overall bipolar illness and the mean change from the preceding phase score for overall bipolar illness was minimal and similar between the 2 treatment groups at all weeks.
The CGI-I mean score was similar between the 2 treatment groups for subjects with schizophrenia and for subjects with bipolar I disorder.
No clinically meaningful difference was demonstrated between the 2 treatment groups for the SWN-S total score for subjects with schizophrenia or bipolar I disorder.

### Overall Conclusions

Disposition: Completion rate at week 32 for Ari 2M group was greater than IM depot. The top three discontinuation reasons for both treatment groups were withdrawal by subjects, adverse event and lost to follow-up.

Safety: Multiple-dose administrations of aripiprazole 2M LAI 960 mg into the gluteal muscle site were generally safe and well tolerated in subjects with schizophrenia or bipolar I disorder and did not show any new safety concerns. The clinical safety of aripiprazole 2M LAI 960 mg relative to aripiprazole IM depot 400 mg was demonstrated with an overall similar incidence of TEAEs and laboratory test abnormalities in both treatment groups. The most frequently reported TEAEs (>10% of subjects in any group) of injection site pain and increased weight are consistent with the known safety profile of aripiprazole.

### Pharmacokinetic:

Aripiprazole 2M LAI 960 mg had a similar aripiprazole AUC₀₋₅₆ following the fourth dose compared to the sum of AUC0-28 following the seventh and eighth dose of aripiprazole IM depot 400 mg (GMR [90% CI (confidence interval)]: 1.006 [0.851, 1.190]).
Aripiprazole 2M LAI 960 mg had a similar aripiprazole C₅₆ following the fourth dose compared to C₂₈ following the eighth dose of aripiprazole IM depot 400 mg (GMR [90% CI]: 1.011 [0.893, 1.145]).

Efficacy: The treatment effects of the aripiprazole 2M LAI 960 mg group were comparable to the aripiprazole IM depot 400 mg group with respect to changes from baseline in the PANSS and CGI-S (for subjects with schizophrenia); MADRS, YMRS, and CGI-BP (for subjects with bipolar I disorder); and CGI-I and SWN-S (for all subjects).

### Example Two

A Phase 1, Open Label, Single Ascending Dose, Parallel Arm Trial to Determine the Pharmacokinetics, Safety, and Tolerability of Aripiprazole 2 Month Intramuscular Depot Administered Gluteally in Adult Subjects with Schizophrenia

This trial was an open-label, single ascending dose, parallel-arm, multiple-center trial to determine the PK, safety, and tolerability of single-dose administration of 780 mg (Cohort 1) and 1200 mg (Cohort 2) of a high dose formulation of aripiprazole 2M RTU that is in the gluteal muscle of adult subjects with schizophrenia. The data from this trial is supportive information. Overall, aripiprazole 2M RTU was well tolerated when administered IM as a single dose of 780 mg and 1200 mg to adult subjects with schizophrenia. In a subset of 17 subjects, the administration of aripiprazole 2M RTU resulted in lasting aripiprazole plasma concentrations, resulting in aripiprazole plasma concentrations were consistently above the mean PK profile following a single gluteal administration of 400 mg Abilify Maintena®. Safety data from this subset of subjects was evaluated and compared to the known safety profile of Abilify Maintena®.

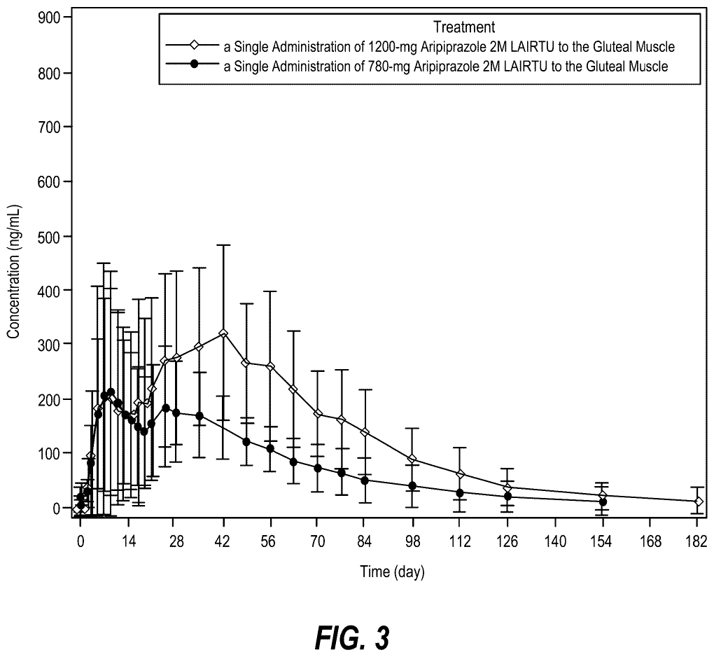

The aripiprazole 2M RTU was an extended-release presentation for dosing every 2 months at the dose levels evaluated. The extension of the dosing interval for the aripiprazole 2M RTU was primarily through an increase in the dose while maintaining minimum aripiprazole concentrations that are comparable to Abilify Maintena® after multiple doses. The aripiprazole 2M RTU was engineered with higher aripiprazole concentrations in the drug product (300 mg/mL vs 200 mg/mL) and minor changes to the vehicle compared to currently marketed/approved Abilify Maintena®. The mean aripiprazole particle size distribution and the dissolution profile for the aripiprazole 2M RTU formulation were comparable with the Abilify Maintena® formulation and the formulation was expected to have a similar extended-release profile compared with the approved Abilify Maintena® formulation. Mean (standard deviation [SD]) aripiprazole plasma concentration time profiles following administration of a single dose of 780 mg or 1200 mg aripiprazole to the gluteal muscle in subjects with schizophrenia are presented in FIG. 3.

A summary of the aripiprazole PK parameters following single-dose administration of a single dose of 780 mg or 1200 mg aripiprazole to the gluteal muscle in subjects with schizophrenia is presented in Table 7 below.

Table 7: Mean (SD) Aripiprazole Pharmacokinetic Parameters Following Administration of a Single Dose of 780 mg or 1200 mg Aripiprazole 2M RTU to the Gluteal Muscle of Subjects with Schizophrenia

TABLE 7

| | Aripiprazole 2M RTU | Aripiprazole 2M RTU |
|---|---|---|
| | LAI | LAI |
| | 780 mg | 1200 mg |
| PK Parameter | (N = 18) | (N = 13) |
| Cmax (ng/mL) | 271 (157) | 391 (200) |
| tmax(day)^{a} | 25.1 (4.07-76.0) | 41.0 (6.09-61.9) |
| AUCt (ng · day/mL) | 12600 (3710) | 23800 (7620) |
| AUC∞ (ng · day/mL) | 13400 (4600)^{b} | 24700 (8080)^{c} |
| t½ (day) | 22.1 (16.5)^{b} | 20.0 (9.2)^{b} |
| CL/F (mL/day/kg) | 763 (299)^{b} | 596 (207)^{c} |
| Cmax/Dose (ng/mL/mg) | 0.347 (0.201) | 0326 (0.167) |
| AUCt/Dose ([ng · day/mL]/mg) | 16.1 (4.75) | 19.8 (6.35) |
| AUC∞/Dose([ng · day/mL]/mg) | 17.2 (5.90)^{b} | 20.6 (6.73)^{c} |

From Table 7, it is noted that AUC^{∞} is area under the concentration-time curve calculated from time zero to infinity; AUCt is area under the concentration-time curve calculated to the last observable concentration at time t; CL/F is apparent clearance of drug from plasma after extravascular administration; RTU is ready to use; tmax is time to maximum (peak) plasma concentration; t1/2 is elimination half-life. Additionally, ^{a}Median (minimum-maximum); ^{b}n=14; and ^{c}n=11.

All publications and patents mentioned herein are hereby incorporated by reference in their entirety as if each individual publication or patent was specifically and individually indicated to be incorporated by reference.

Claims or descriptions that include “or” or “and/or” between at least one members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all the group members are present in, employed in, or otherwise relevant to a given product or process.

Furthermore, the disclosure encompasses all variations, combinations, and permutations in which at least one limitation, element, clause, and descriptive term from at least one of the listed claims is introduced into another claim. For example, any claim that is dependent on another claim can be modified to include at least one limitation found in any other claim that is dependent on the same base claim. Where elements are presented as lists, e.g., in Markush group format, each subgroup of the elements is also disclosed, and any element(s) can be removed from the group. It should be understood that, in general, where the disclosure, or aspects of the disclosure, is/are referred to as comprising particular elements and/or features, embodiments of the disclosure or aspects of the disclosure consist, or consist essentially of, such elements and/or features. For purposes of simplicity, those embodiments have not been specifically set forth in haec verba herein. Where ranges are given, endpoints are included. Furthermore, unless otherwise indicated or otherwise evident from the context and understanding of one of ordinary skill in the art, values that are expressed as ranges can assume any specific value or sub range within the stated ranges in different embodiments of the disclosure, to the tenth of the unit of the lower limit of the range, unless the context clearly dictates otherwise.

Those of ordinary skill in the art will recognize or be able to ascertain using no more than routine experimentation, many equivalents to the specific embodiments of the disclosure described herein. Such equivalents are intended to be encompassed by the following claims.

1.-24. (canceled)

25. A method of treating a patient with schizophrenia or bipolar I disorder comprising:
administering to the patient at a gluteal muscle an injectable preparation comprising aripiprazole or a salt thereof,
wherein the patient is administered the injectable preparation once every about 8 weeks or once every about two months, and the amount of aripiprazole in the injectable preparation is about 960 mg.

26. The method according to claim 25, wherein the injectable preparation further comprises: water, and a suspending agent comprising polyvinylpyrrolidone, polyethylene glycol, and carboxymethylcellulose or a salt thereof, wherein aripiprazole or a salt thereof has a mean primary particle diameter ranging from about 0.5 μm to about 30 μm, and the concentration of aripiprazole in the injectable preparation is about 300 mg/mL.

27. The method according to claim 25, wherein the injectable preparation further comprises: water for injection, and a suspending agent comprising polyvinylpyrrolidone, polyethylene glycol, and carboxymethylcellulose or a salt thereof, wherein the aripiprazole or a salt thereof is aripiprazole monohydrate, the aripiprazole monohydrate has a mean primary particle diameter ranging from about 2 μm to about 5 μm, and the concentration of aripiprazole in the injectable preparation is about 300 mg/mL, calculated based on an anhydrous form of aripiprazole.

28. The method according to claim 25, wherein the injectable preparation is in a dosage form of a prefilled sol-gel formed injection.

29. The method according to claim 25, wherein the injectable preparation has a thixotropic property.

30. The method according to claim 25, further comprising administering a plurality of the injectable preparations to the patient, wherein the plurality of the injectable preparations results in an area under a concentration-time curve (AUC) of aripiprazole from time zero to 56 days postdose substantially equal to a twofold AUC of aripiprazole from time zero to 28 days postdose following a plurality of administration of an aripiprazole intramuscular (IM) depot formulation

31. The method of according to claim 25, further comprising administering aripiprazole or a salt thereof in a solid dosage form orally, and the solid dosage form is administered in an amount ranging from 10 mg to 20 mg per day consecutively for 5 days to 15 days with the administration of the injectable preparation.

32. The method according to claim 25, further comprising administering from 10 mg to 20 mg of aripiprazole or a salt thereof in a solid dosage form orally per day consecutively for 7 days with the administration of the injectable preparation.

33. The method according to claim 30, wherein the consecutive administrations allows for a mean plasma level of aripiprazole in the patient to maintain 90 ng/mL or more.

34. The method according to claim 32, wherein the method does not comprise a further oral administration of aripiprazole or a salt thereof.

35. A method of administering an injectable preparation comprising aripiprazole or a salt thereof to a patient with schizophrenia or bipolar I disorder comprising:
administering orally to the patient from 10 mg/day to 20 mg/day of aripiprazole or a salt thereof consecutively for 5 days to 15 days from a first day;
administering intramuscularly to the patient the injectable preparation once every about 56 days,
wherein the injectable preparation comprises about 960 mg of aripiprazole or a salt thereof and is administered on the first day, and the consecutive administrations allows for a mean plasma level of aripiprazole in the patient to maintain 90 ng/mL or more.

36. The method according to claim 35, wherein the method does not comprise a further oral administration of aripiprazole or a salt thereof.

37. The method according to claim 35, wherein the injectable preparation further comprises: water, and a suspending agent comprising
polyvinylpyrrolidone, polyethylene glycol, and carboxymethylcellulose or a salt thereof, wherein aripiprazole or a salt thereof has a mean primary particle diameter ranging from about 0.5 μm to about 30 μm, and the concentration of aripiprazole in the injectable preparation is about 300 mg/mL.

38. The method according to claim 35, wherein the injectable preparation further comprises: water for injection, and a suspending agent comprising polyvinylpyrrolidone, polyethylene glycol, and carboxymethylcellulose or a salt thereof, wherein the aripiprazole or a salt thereof is aripiprazole monohydrate, the aripiprazole monohydrate has a mean primary particle diameter ranging from about 2 μm to about 5 μm, and the concentration of aripiprazole in the injectable preparation is about 300 mg/mL, calculated based on an anhydrous form of aripiprazole.

39. The method according to claim 35, wherein the injectable preparation is in a dosage form of a prefilled sol-gel formed injection.

40. The method according to claim 35, wherein the injectable preparation has a thixotropic property.

41. The method according to claim 25, wherein the patient is administered the injectable preparation once every about 56 days.
